(19) United States
(12) Patent Application Publication
(10) Pub. No.: US 2008/0172010 A1
(43) Pub. Date: Jul. 17, 2008

(54) Disposable Endoscope Connector

(75) Inventors: Boris Degtiar, Modi'in (IL); Eran Shor, Bitzaron (IL)

Correspondence Address: DARBY & DARBY P.C., P.O. BOX 770, Church Street Station, New York, NY, 10008-0770, US

(73) Assignee: G.I. VIEW LTD., Ramat Gan (IL)

(21) Appl. No.: 11/914,089

(22) PCT Filed: May 11, 2006
(86) PCT No.: PCT/IL06/00561

§ 371 Date: Jan. 2, 2008

Related U.S. Application Data
(60) Provisional application No. 60/680,074, filed on May 11, 2005.

(51) Int. Cl. A61M 31/00

(52) U.S. Cl. 604/264

(57) ABSTRACT

Apparatus is provided, including an elongate carrier (26), adapted to be inserted through a proximal opening of a body lumen (20), and a workstation (402), which includes a generator of negative fluid pressure. A disposable connector (400) is adapted to be coupled to the elongate carrier, the connector including a filter (412) and a tube (408) coupled to the filter, the tube being adapted to be coupled to the negative fluid pressure.

### CROSS-REFERENCES TO RELATED APPLICATIONS

The present patent application claims priority from U.S. Provisional Patent Application 60/680,074 to Degtiar et al., filed May 11, 2005, entitled, “Disposable endoscope connector,” which is assigned to the assignee of the present patent application and is incorporated herein by reference.

### FIELD OF THE INVENTION

The present invention relates generally to endoscopic systems, such as for examination of the gastrointestinal (GI) tract.

### BACKGROUND OF THE INVENTION

The following references are incorporated herein by reference:

U.S. Pat. No. 5,337,732 to Grundfest et al.

US Patent Application Publication 2003/0168068 to Poole and Young

US Patent Application Publication 2003/0105386 and U.S. Pat. No. 6,485,409 to Voloshin et al.

US Patent Application Puiblication 2002/0107478 to Wendlandt

U.S. Pat. No. 6,702,735 to Kelly

U.S. Pat. No. 5,259,364 to Bob, et al.

U.S. Pat. No. 4,403,985 to Boretos

U.S. Pat. No. 4,176,662 to Frazer

U.S. Pat. No. 4,148,307 to Utsugi

U.S. Pat. No. 5,906,591 to Dario et al.

U.S. Pat. No. 6,007,482 to Madni et al.

U.S. Pat. No. 5,662,587 to Gimndfest et al.

U.S. Pat. No. 4,690,131 to Lyddy, Jr. et al.

U.S. Pat. No. 4,040,413 to Ohshiro

U.S. Pat. No. 6,503,192 to Ouchi

US Patent Application Publication 2003/0083547 to Hamilton et al.

PCT Publication WO 04/069057 to Gobel

US Patent Application Publication 2003/0000526 to Gobel

PCT Publication WO 03/045487 to Gobel

### SUMMARY OF THE INVENTION

Some embodiments of the present invention provide an imaging system which is propelled by fluid pressure through a body lumen, such as the gastrointestinal (GI) tract. Embodiments of the invention are described hereinbelow with reference to the GI tract, but it is understood that these embodiments are not limited to use in the GI tract, and may be used for other body lumens as well. Unlike the prior art, which may inflate and anchor balloons and similar devices to the GI tract wall in an attempt to overcome the low friction of the GI tract, these embodiments of the present invention utilize the very low friction environment of the GI tract to propel the imaging system, typically with no need for anchoring.

There is thus provided, in accordance with an embodiment of the present invention, a system including a guide member at least partially insertable into a proximal opening of a body lumen, the guide member including a first passageway connectable to a source of fluid pressure, an elongate carrier arranged for sliding movement through the guide member, and a piston head mounted on the carrier, wherein a greater fluid pressure acting on a proximal side of the piston head than on a distal side of the piston head propels the piston head and the carrier in a distal direction in the body lumen.

The system of this embodiment of the invention may have different features. For example, the piston head may be inflatable. The carrier may include a second passageway in fluid communication with the piston head, which may be connected to a source of fluid pressure for inflating the piston head. A vent tube may pass through the piston head, having an opening distal to the piston head through which fluid may be vented to the outside. An image-capturing device may be mounted on the carrier, such as distal to the piston head. A power supply tube may pass through the carrier and may be connected to the image-capturing device. A fluid supply tube may pass through the carrier and may be connected to a fluid source.

In accordance with an embodiment of the present invention, an auxiliary piston head may be mounted on the carrier proximal to the first-mentioned piston head. The auxiliary piston head, which may be inflatable, may be fixed axially to the carrier at a fixed or variable distance from the first-mentioned piston head. The carrier may include a third passageway in fluid communication with the auxiliary piston head, which may be connected to a source of fluid pressure for inflating the auxiliary piston head.

In some embodiments of the present invention, a disposable connector is provided for coupling the endoscopic imaging system with an external workstation. The disposable connector comprises one or more filters that are configured to filter fluid (i.e., liquid or gas) passing between the imaging system and the external workstation. For some applications, the one or more filters comprise a suction trap that comprises a container for containing fluid from the body that is not supposed to enter the external workstation. The use of such a disposable connector obviates the need to periodically replace filters in the workstation and/or in a fluid supply line coupled to the imaging system.

There is therefore provided, in accordance with an embodiment of the present invention, apparatus for use with a biologically-compatible-fluid pressure source, including:

an elongate carrier, adapted to be inserted through a proximal opening of a body lumen; and

a distal piston head coupled to a distal portion of the carrier and adapted to:
be in direct contact with a wall of the lumen when the carrier is inserted into the lumen,
be advanced distally through the body lumen in response to pressure from the fluid pressure source, and

facilitate passage of fluid out of the lumen from a site within the lumen distal to the piston head.

There is also provided, in accordance with an embodiment of the present invention, apparatus including:

an elongate carrier, adapted to be inserted through a proximal opening of a body lumen;

a workstation, which includes a generator of negative fluid pressure; and

a disposable connector, adapted to be coupled to the elongate carrier, the connector including a filter and a tube coupled to the filter, the tube adapted to be coupled to the negative fluid pressure.

In an embodiment, the filter includes a suction trap including a container for containing fluid from the body lumen.

In an embodiment, the lumen includes a gastrointestinal (GI) tract, and wherein the carrier is adapted to be inserted through the proximal opening of the GI tract.

In an embodiment, the GI tract includes a colon, and wherein the carrier is adapted to be inserted through the proximal opening of the colon.

In an embodiment, the workstation includes a source of positive fluid pressure, and wherein the elongate carrier includes a piston head coupled to a distal portion of the carrier and adapted to:

form a pressure seal with a wall of the lumen after the carrier has been inserted into the lumen, and

be advanced distally through the body lumen in response to pressure from the positive fluid pressure source.

In an embodiment, the disposable connector is adapted to be fixedly coupled to the elongate carrier.

In an embodiment, the disposable connector is adapted to be removably coupled to the elongate carrier.

### BRIEF DESCRIPTION OF THE DRAWINGS

The present invention will be understood and appreciated more fully from the following detailed description taken in conjunction with the drawings in which:

FIG. 1 is a simplified pictorial illustration of a system, constructed and operative in accordance with an embodiment of the present invention, which may be suitable for imaging body lumens, such as the GI tract;

FIGS. 2 and 3 are simplified sectional illustrations of distal and proximal portions, respectively, of the system of FIG. 1;

FIG. 4 is a simplified sectional illustration of a carrier of the system of FIG. 1, the section being taken transverse to a longitudinal axis of the carrier, in accordance with an embodiment of the present invention;

FIGS. 5A, 5B and 5C are simplified pictorial illustrations of the system of FIG. 1, showing three steps of a mode of operation thereof, wherein inflatable piston heads are inflated and deflated to negotiate obstacles in a body lumen, in accordance with an embodiment of the present invention;

FIG. 6 is a pictorial illustration of a system for use in a body lumen, constructed and operative in accordance with an embodiment of the present invention;

FIG. 7 is a pictorial illustration of an inflated conical balloon, which is adapted for use in accordance with an embodiment of the present invention;

FIG. 8 is a pictorial illustration of a partially-inflated conical balloon in a body lumen, in accordance with an embodiment of the present invention;

FIG. 9A is a pictorial illustration of the cross-section of a fully inflated portion of a conical balloon, in accordance with an embodiment of the present invention;

FIG. 9B is a pictorial illustration of the cross-section of a partially inflated portion of a conical balloon, in accordance with an embodiment of the present invention;

FIGS. 10A and 10B are pictorial illustrations of a system for use in a body lumen, constructed and operative in accordance with an embodiment of the present invention;

FIGS. 11A and 11B are pictorial illustrations of the multi-lobed-piston head of FIGS. 10A and 10B, in accordance with an embodiment of the present invention;

FIG. 12 is a schematic cross-sectional illustration of an optical system, in accordance with an embodiment of the present invention;

FIGS. 13A and 13B are pictorial illustrations of another system for use in a body lumen, in accordance with an embodiment of the present invention;

FIG. 14 is a schematic illustration of an inserter, in accordance with an embodiment of the present invention;

FIG. 15 is a schematic illustration of a cleaning system, in accordance with an embodiment of the present invention; and

FIG. 16 is a schematic illustration of a disposable connector, in accordance with an embodiment of the present invention.

### DETAILED DESCRIPTION OF EMBODIMENTS

Reference is now made to FIGS. 1-3, which illustrate a system 10, constructed and operative in accordance with an embodiment of the present invention.

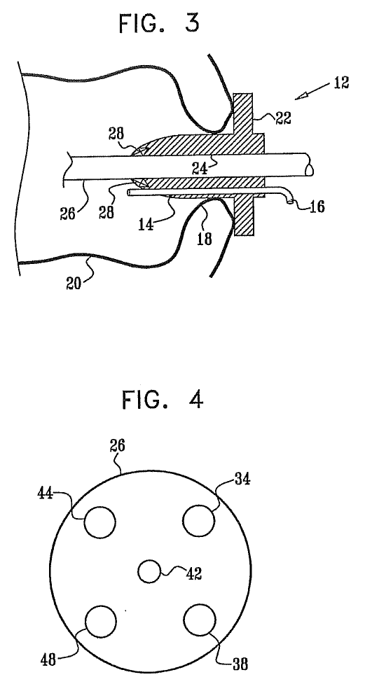

As seen best in FIG. 3, system 10 may include a guide member 12, which may be constructed of any medically safe material, such as but not limited to, plastic or metal. Guide member 12 may be formed with a first passageway 14 connected to a source 16 of a pressurized biologically-compatible fluid (“fluid pressure source 16”), such as but not limited to, a source of pressurized air, CO₂ or water. Guide member 12 may be at least partially insertable into a proximal opening 18 (e.g., the rectum) of a body lumen 20 (e.g., the colon). Guide member 12 may include an annular ring 22 for abutting against the proximal opening 18.

Guide member 12 may be formed with a bore 24 through which an elongate carrier 26 may be arranged for sliding movement. An O-ring 28 may be provided for dynamically sealing carrier 26 in its sliding motion relative to the guide member 12. Carrier 26 may be any slender wire, catheter or tube and the like, constructed of any medically safe material, such as but not limited to, a flexible plastic or metal. Carrier 26, including its tip, may be safely deflected and steered through body lumen 20.

In an embodiment of the present invention, guide member 12 comprises a microcuff, which forms a seal with the wall of lumen 20, in order to maintain positive pressure within lumen 20. For example, the microcuff may comprise a cuff manufactured by Microcuff GmbH (Weinheim, Germany), and/or described in the above-mentioned PCT Publication WO 04/069057, US Patent Application Publication 2003/0000526, and/or PCT Publication WO 03/045487. The creation of such positive pressure is described hereinbelow.

A piston head 30 may be mounted on carrier 26. Piston head 30 may be inflatable, and as such may be constructed of any medically safe elastomeric material, such as but not limited to, a bladder or membrane made of polyurethane or silicone rubber, for example. Alternatively, for some applications, piston head 30 is not inflatable, and is configured to maintain a seal with the wall of lumen 20, while at the same time allowing the piston head to advance in the lumen. For example, the piston head may comprise a flexible, resilient material, shaped like a cup or an umbrella. In an embodiment, the non-inflatable piston head is controllable to be in a first configuration during advancement, during which time the seal is maintained, and in a second configuration during withdrawal, during which time the seal is broken.

An image-capturing device 32 may be mounted on carrier 26 distal to piston head 30. Piston head 30 is typically fixed to carrier 26 and sealed thereto with O-rings 33, but optionally may be arranged to slide on carrier 26 up to some distal stop which arrests further distal motion of piston head 30 (image-capturing device 32 may serve as the distal stop, for example). Image-capturing device 32 may comprise, without limitation, a camera (e.g., CCD or CMOS), or alternatively x-ray, ultrasonic, MRI, infrared and/or microwave imaging devices.

Other therapeutic or diagnostic devices may be mounted on or in carrier 26, such as but not limited to, a magnet, drug delivery devices (e.g., via iontophoresis), gene therapy devices and others.

Carrier 26 may include a second passageway 34 in fluid communication with piston head 30, connected to a source of fluid pressure 36 (e.g., pressurized air or water) for inflating piston head 30. For some applications, piston head-inflation fluid pressure source 36 is regulated to maintain a generally constant pressure within piston head 30, regardless of changes of volume of the piston head which occur in response to diameter changes of lumen 20.

A vent tube 38 may pass through or around piston head 30, having an opening 40 distal to piston head 30 through which fluid is ventable to the outside. That is, the proximal end of vent tube 38 vents the fluid past guide member 12 to the outside. For some applications, the proximal end of vent tube 38 may be connected to a suction source (not shown) for sucking fluid through vent tube 38. “Fluid,” as used herein, including in the claims, includes liquids and gases.

In an embodiment, vent tube 38 is not used, but instead piston head 30 is temporarily deflated (at least in part), intermittently and/or in response to excess pressure accumulating distal to piston head 30. The temporary deflation of the piston head allows venting of the distal pressure to occur through passageway 14, typically in conjunction with a temporary decoupling of passageway 14 from fluid pressure source 16.

A power supply tube 42 (e.g., containing electrical wires, fiber optics, etc.) may pass through carrier 26, for connection to image-capturing device 32. Alternatively, the electrical and optical components of image-capturing device 32 may have their own internal power source, with no need for external wiring. Image-capturing device 32 may wirelessly transmit or receive data to or from an external processor (not shown). The components of system 10 may be fully automated with sensors and operate in a closed or open control loop.

A fluid supply tube 44 may pass through carrier 26, which may be connected to a fluid source (not shown), e.g., pressurized water, for cleaning the area near image-capturing device 32, or in combination with the vent tube 38, for cleaning body lumen 20 itself (e.g., the colon).

Experiments carried out by the inventors have shown that the system, as described hereinabove, is able to safely and efficiently advance a colonoscope or other tool through the colon of an anesthetized 90 kg pig. In these experiments, elongate carrier 26 was generally radio-opaque, and its motion was tracked in real-time using fluoroscopic imaging. Vent tube 38 was utilized, having an inner diameter of 2 mm. It acted passively (without being connected to a suction source), in order to allow pressure accumulating distal to piston head 30 to be vented to the outside.

In these experiments, a range of operating pressures were examined. The proximal pressure and the pressure within the piston head (intra-head pressure) were controlled, and values were recorded at which satisfactory movement of piston head 30 was observed. In general, for intra-head pressures ranging between 25 and 40 millibar, movement of piston head 30 was observed when the proximal pressure reached 30-100% of the intra-head pressure.

Typically, when the proximal pressure was below a threshold value, no movement was observed. As the proximal pressure was elevated above the threshold value, piston head 30 advanced through the colon. If the proximal pressure increased significantly above the threshold pressure (e.g., 2-10 millibar above the threshold pressure), then there was pressure leakage between piston head 30 and the wall of lumen 20, and advancement of piston head 30 ceased. In response to such a leak, the proximal pressure was lowered, vent tube 38 allowed the excess accumulated distal pressure to vent to the outside, and movement of piston head 30 recommenced.

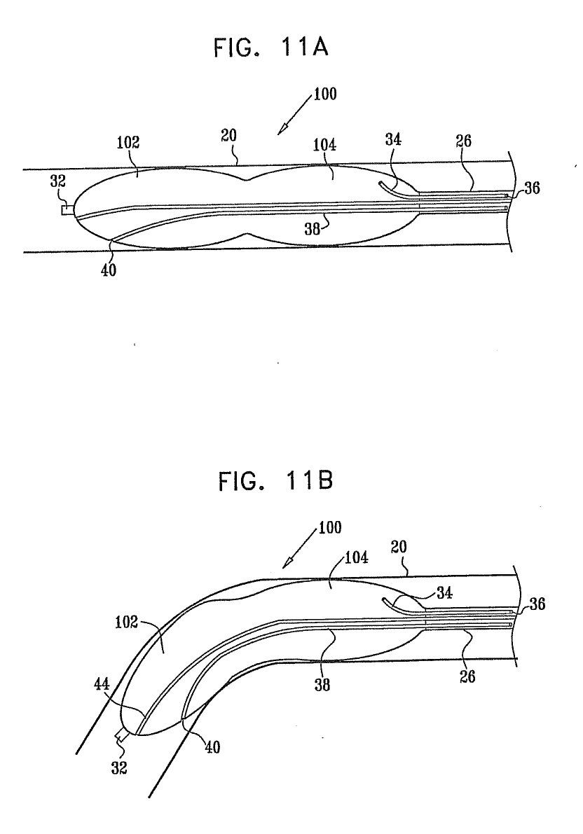

In an experiment, an inflatable piston head was formed of thin silicone, and was shaped to have a distal lobe, a proximal lobe, and an intermediate portion connecting the distal and proximal lobes. (See FIGS. 10A and 10B.) For an intra-head pressure of 30 millibar, the piston head advanced through the colon when the proximal pressure was maintained between 10 and 20 millibar. During advancement of the piston head, vent tube 38 vented to the outside the pressure that accumulated due to the advancement of the piston head. Leakage around the piston head was observed for proximal pressures greater than about 20 millibar. For an intra-head pressure of 40 millibar, the piston head advanced through the colon when the proximal pressure was maintained between 27 and 30 millibar, both on straight and curved portions of the colon. For straight portions of the colon, proximal pressures of as low as 20 millibar were also sufficient to produce satisfactory movement of the piston head.

Although the rate of advance of the two-lobed piston head was found to vary with the selected pressures, in one experiment using a thin-walled two-lobed piston head, a total time of 2 minutes passed during the advancing of a colonoscope 1.5 meters into the colon of the pig. In another experiment, using a thick-walled two-lobed piston head, an intra-head pressure of 70 millibar and proximal pressure of 50 millibar resulted in 1.5 meters of colonoscope advancement in 1 minute 41 seconds. Thin-walled piston heads useful for these embodiments of the invention typically have a head wall thickness between 10 and 100 microns, e.g., 50 microns or less than 20 microns, or a head wall thickness of less than 10 microns. Thick-walled piston heads useful for these embodiments of the invention typically have a head wall thickness greater than 100 microns, e.g., 150 microns, or 250 microns.

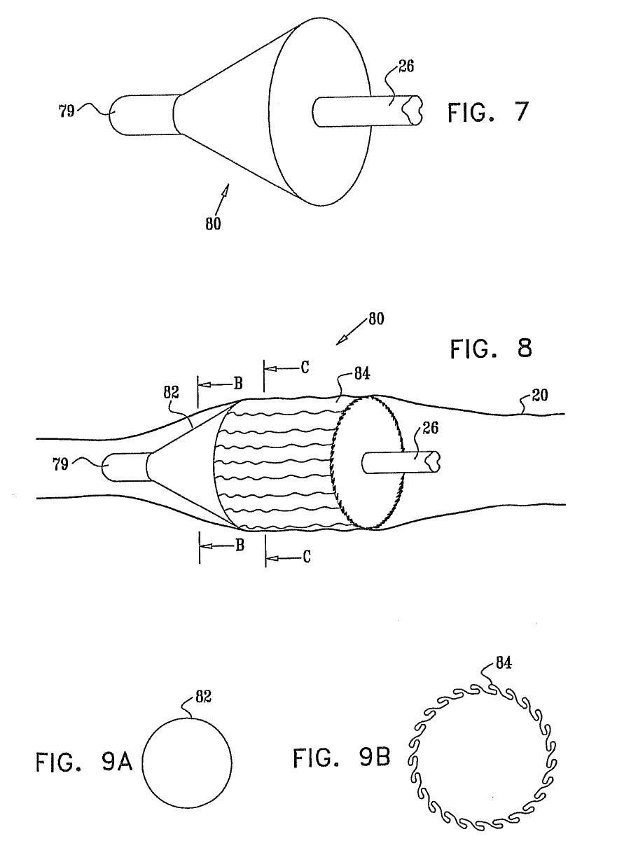

In another experiment, the piston head was formed of polyurethane, and was shaped like a cone, as described hereinbelow with reference to FIGS. 7-9. In this experiment, satisfactory advancement of the piston head was obtained at a proximal pressure of 35 millibar, when the intra-head pressure was also 35 millibar. The satisfactory advancement was obtained both on straight and curved portions of the colon.

It is noted that in these experiments, during the time when the intra-head pressure was kept constant, the volume of the piston head changed actively in response to changes in diameter of lumen 20.

Reference is now made to FIGS. 1, 2 and 5A-C, which illustrate operation of system 10, in accordance with an embodiment of the present invention. In this embodiment, an auxiliary piston head 46 may be mounted on the carrier proximal to distal piston head 30. Auxiliary piston head 46, which like piston head 30 may be inflatable, may be fixed axially to carrier 26 at a fixed distance from piston head 30. Auxiliary piston head 46 may be sealed with respect to carrier 26 with O-rings 47. Carrier 26 may include a third passageway 48 in fluid communication with auxiliary piston head 46, connected to a source of fluid pressure 50 for inflating auxiliary piston head 46.

System 10 may be inserted in the rectum with piston heads 30 and 46 initially deflated to facilitate insertion. Distal piston head 30 may then be gently inflated until it expands to the inner wall of body lumen 20. This configuration is shown in FIG. 1. Pressurized fluid (e.g., air) from fluid pressure source 16 may be introduced into the colon through the first passageway 14 of guide member 12. The pressurized fluid creates greater fluid pressure acting on the proximal side of piston head 30 than on the distal side of piston head 30. Opening 40 of vent tube 38 may assist in creating the pressure difference across piston head 30, either passively, or actively via applied suction. This pressure difference propels piston head 30 together with carrier 26 distally into the body lumen (in this example, the colon), as indicated by arrow 60. Image-capturing device 32 may capture images of body lumen 20 as system 10 travels therethrough.

In an embodiment of the present invention, the techniques described herein for propulsion by creating a pressure difference are applied in a reverse manner to actively propel piston head 30 together with carrier 26 proximally, i.e., to withdraw system 10 from lumen 20. Pressurized fluid (e.g., air) from a fluid pressure source is introduced to the distal side of piston head 30, via a pressure-application tube passing through or around piston head 30. Optionally, vent tube 38 serves as the pressure-application tube during withdrawal. The pressurized fluid creates greater fluid pressure acting on the distal side of piston head 30 than on the proximal side of piston head 30, thereby proximally propelling the piston head and the carrier. A vent tube between the proximal side of piston head 30 and outside the lumen may assist in creating the pressure difference across piston head 30, either passively, or actively via applied suction. Optionally, passageway 14 serves as the vent tube during withdrawal.

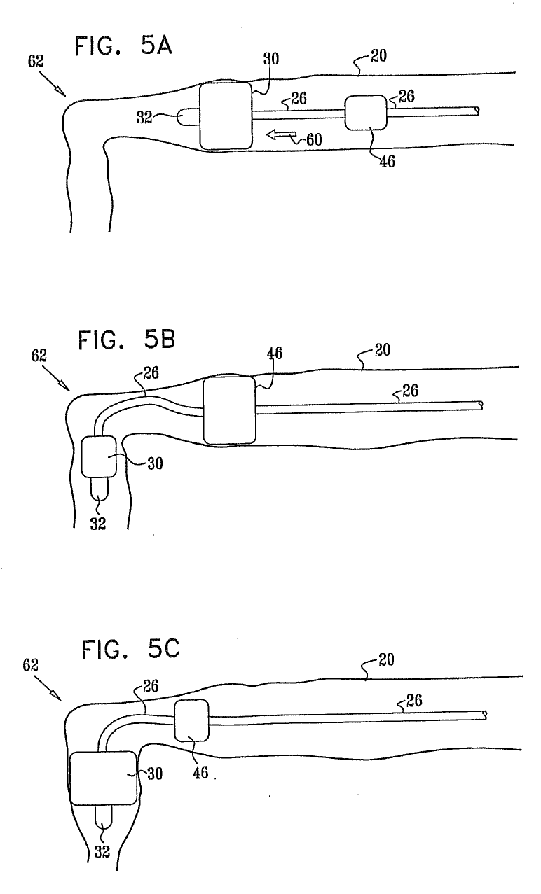

As seen in FIG. 5A, system 10 may eventually reach an obstacle or tight turn, indicated by arrow 62. In such a case, proximal piston head 46 may be inflated and distal piston head 30 may be deflated as shown in FIG. 5B. In this configuration, the pressurized fluid creates greater fluid pressure acting on the proximal side of proximal piston head 46 than on the distal side of proximal piston head 46. This pressure difference propels proximal piston head 46 together with carrier 26 distally, as indicated by arrow 64. This distal movement brings distal deflated piston head 30 past the obstacle, as seen in FIG. 5B. System 10 continues its distal movement in body lumen 20 until proximal piston head 46 reaches the obstacle. At this point, distal piston head 30 may be inflated and proximal piston head 46 may be deflated once again, as shown in FIG. 5C. Once again, the pressurized fluid creates greater fluid pressure acting on the proximal side of distal piston head 30 than on the distal side of distal piston head 30. The pressure difference propels system 10 distally in body lumen 20, and brings proximal deflated piston head 46 past the obstacle. The cycle may be repeated as often as necessary.

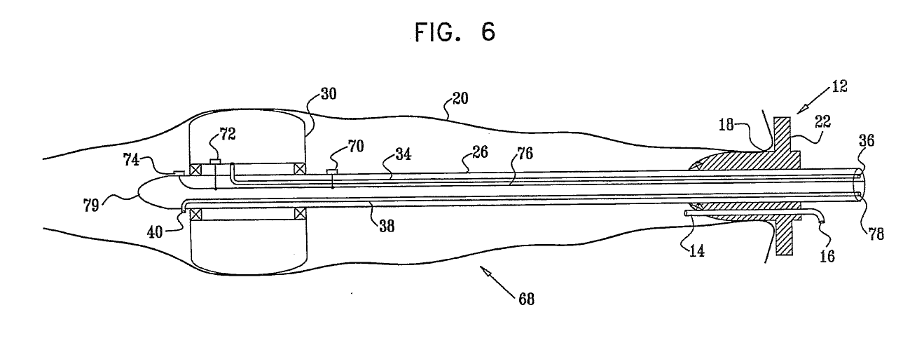

Reference is now made to FIG. 6, which illustrates a system 68, constructed and operative in accordance with an embodiment of the present invention. System 68 operates in substantially the same manner as system 10, described hereinabove with reference to FIGS. 1-4, in that distal piston head 30 is inflated until it is in contact with body lumen 20, such that a seal between piston head 30 and lumen 20 is formed. Pressurized fluid is then introduced via first passageway 14, producing a larger pressure on the proximal face of piston head 30 than on the distal face of piston head 30, resulting in a net force acting to move piston head 30 distally. A sufficient net pressure force results in distal movement of piston head 30 along with elongate carrier 26 and a tool 79. Tool 79 may comprise an imaging device, a biopsy device, or other apparatus to be used in body lumen 20.

Additionally, for some applications of the present invention, a suction source 78 is coupled to opening 40 via vent tube 38 to provide suction on the distal face of piston head 30 and facilitate the distal movement of piston head 30. Providing suction at opening 40 may also be used in some applications to remove contents of the lumen, such as excess fluid or stool, that are impeding the movement of piston head 30. For some applications, the suction decreases an accumulation of gas distal to piston head 30 that may be uncomfortable for the patient.

System 68 typically comprises one or more pressure sensors, for example in order to be able to improve or optimize the performance of the system with respect to ease and speed of movement of system 68 through lumen 20. In particular, system 68 typically comprises one or more of the following pressure sensors:
a first pressure sensor 70, adapted to determine the pressure acting on the proximal face of distal piston 30;
a second pressure sensor 72, adapted to determine the inflation pressure of the distal piston head; and/or
a third pressure sensor 74, adapted to determine the pressure acting on the distal face of piston head 30.

For some applications, the three pressure sensors are coupled to a pressure sensor bus 76, such that the various pressure readings can be sent to an electromechanical or mechanical control unit (not shown), which regulates the different pressures, either automatically or with input from the operator of the system. For some applications, only one of the pressure sensors is included in system 68 (e.g., sensor 70, sensor 72, or sensor 74). For other applications, two of the pressure sensors are included, and one is omitted (e.g., sensor 70, sensor 72, or sensor 74).

For some applications, first pressure sensor 70 is located proximal to distal piston head 30 in a vicinity of the piston head. Alternatively, first pressure sensor 70 is located in a vicinity of fluid pressure source 16, typically outside the body of the patient. In this latter configuration: (a) first pressure sensor 70 is integrated with pressure source 16, or is positioned separately from pressure source 16; and (b) first pressure sensor 70 is in fluid communication with a proximal portion of lumen 20 proximal to piston head 30, either via first passageway 14, or via a separate passageway in fluid communication with first pressure sensor 70 and the proximal portion of lumen 20 (separate passageway not shown). A distal end of such separate passageway is adapted to be positioned in the proximal portion of lumen 20, either in a vicinity of guide member 12, or more distally in lumen 20, such as in a vicinity of piston head 30 proximal to the piston head.

For some applications, second pressure sensor 72 is located inside distal piston head 30. Alternatively, second pressure sensor 72 is located in a vicinity of fluid pressure source 36, typically outside the body of the patient. In this latter configuration, second pressure sensor 72 is in fluid communication with piston head 30, either via second passageway 34, or via a separate passageway in fluid communication with second pressure sensor 72 and piston head 30 (separate passageway not shown).

For some applications, third pressure sensor 74 is located distal to distal piston head 30. Alternatively, third pressure sensor 74 is located in a vicinity of a proximal opening of vent tube 38 (which, for applications in which suction source 78 is provided, is in a vicinity of the suction source), typically outside the body of the patient. In this latter configuration: (a) third pressure sensor 74 is integrated with suction source 78, or is positioned separately from suction source 78; and (b) third pressure sensor 74 is in fluid communication with a distal portion of lumen 20 distal to piston head 30, either via vent tube 38, or via a separate passageway in fluid communication with third pressure sensor 72 and the distal portion of lumen 20 (separate passageway not shown).

For some applications in which third pressure sensor 78 is in fluid communication with the distal portion of lumen 20 via vent tube 38, a source such as suction source 78 is adapted to periodically, such as once every 5 to 15 seconds, e.g., once every 10 seconds, generate a burst of fluid (i.e., liquid or gas) in vent tube 38, in order to clear from the tube any bodily material which may have entered the tube through opening 40. Similarly, for some applications in which third pressure sensor 78 is in fluid communication with the distal portion of lumen 20 via a separate passageway, an additional source of pressure coupled to a proximal end of the separate passageway periodically generates a burst of fluid in the separate passageway.

In some embodiments of the present invention, satisfactory performance of system 68 is attained by maintaining a pressure on the proximal side of piston head 30 at about 25 millibar gauge, a pressure on the distal side of piston head 30 at about 5 millibar gauge, and a pressure inside piston head 30 at about 20 millibar gauge. These values typically range, as appropriate, between about +10 and +50 millibar, −5 and +15 millibar, and +10 and +60 millibar, respectively.

For some applications, during distal advancement of system 68, the pressure inside piston head 30 is maintained within about 5 millibar of the pressure differential across either side of piston head 30. For example, using the exemplary numbers cited above, a pressured differential across the piston head is 25 millibar−5 millibar=20 millibar. By maintaining the pressure inside piston head 30 within 5 millibar of the pressure differential, the pressure inside piston head 30 would generally remain between 15 and 25 millibar. The pressure within piston head 30 is typically maintained near this differential pressure when piston head 30 comprises a flexible but substantially non-elastic material (e.g., a material such as a polyurethane that stretches less than 10% during inflation at less than 50 millibar). For embodiments in which piston head 30 comprises a flexible and elastic material (e.g., a material comprising silicone that stretches more than 10% during inflation at less than 50 millibar), the pressure within piston head 30 is typically greater than the differential pressure.

In an embodiment of the present invention, during distal advancement of system 68, the pressure inside piston head 30 is set to an initial value, such as between about 5 and 15 millibar, e.g., about 10 millibar. The pressure on the proximal side of piston head 30 is increased, typically gradually, and, simultaneously, the pressure inside piston head 30 is regulated to be the greater of (a) its initial value and (b) the pressure on the proximal side of piston head 30 plus a value such as a constant value. Typically, this constant value is between about 1 and about 5 millibar, e.g., between about 1.5 and about 2 millibar, such as about 2 millibar. Once system 68 begins advancing distally, the pressure on proximal head 30 generally declines or remains level, despite the continuous application of pressure by pressure source 16. A diameter of first passageway 14 is typically of a value sufficiently small to limit the increase over time of the pressure proximal to piston head 30 when system 68 is advancing distally. For example, the diameter of first passageway may be between about 3 and about 6 mm. In general, in this embodiment, substantially real-time control of the pressure in piston head 30 is exercised, while real-time control of the pressure in lumen 20 proximal to the piston head is not necessarily exercised.

Other combinations of the distal, proximal, and inside pressures for piston head 30 may be better suited for some applications, and the above numbers are not meant to limit the various operating pressures of embodiments of the current invention. Additionally, for some applications of the present invention, the various pressures acting on piston head 30 are regulated depending on where in the lumen the piston head is located.

Although FIG. 6 only shows a distal piston head, it is to be understood that the scope of the present invention includes a system comprising a proximal piston head, as shown in FIG. 1, comprising the various pressure control and measuring apparatus described hereinabove with regard to distal piston head 30 of FIG. 6.

Reference is now made to FIG. 7, which illustrates an inflatable piston head 80, constructed and operative in accordance with an embodiment of the present invention. Inflatable piston head 80 comprises an inflatable balloon that has the general form of a body of revolution about the axis formed by elongate carrier 26, wherein the distal end has a smaller diameter than the proximal end. Piston head 80 typically comprises a material that is flexible but substantially inelastic in the range of pressures typically encountered, such that the shape of the piston head is not substantially changed by elastic deformation when the piston head is inflated. Alternatively, piston head 80 comprises a flexible and elastic material. In some embodiments of the present invention, inflatable piston head 80 has the shape of a cone, as shown in FIG. 7. It is noted that whereas a cone is formed by rotating a straight line about an axis of revolution, other shapes for inflatable piston head 80 are formed by rotating curved lines about an axis of revolution. For example, a parabola or circular arc may be used to generate appropriate shapes. In the context of the present patent application and in the claims, all such shapes which become narrower towards their distal end are referred to as having a “distally-narrowing portion.”

For some embodiments of the present invention, the base of inflatable piston head 80 is flat. In some other embodiments, the base of inflatable piston head 80 is curved, wherein the curvature may be either concave or convex.

FIG. 8 shows an application of inflatable piston head 80, in accordance with an embodiment of the present invention. Piston head 80 is typically inserted into lumen 20 in a deflated state and subsequently inflated until appropriate contact is made with the lumen. Due to the shape of inflatable piston head 80, most of a fully-inflated portion 82 of the piston head is not in substantial contact with lumen 20, while a partially-inflated portion 84 of the piston head is in contact with lumen 20, once the piston head is fully pressurized. A good seal between piston head 80 and lumen 20 is typically obtained where fully-inflated portion 82 meets partially-inflated portion 84.

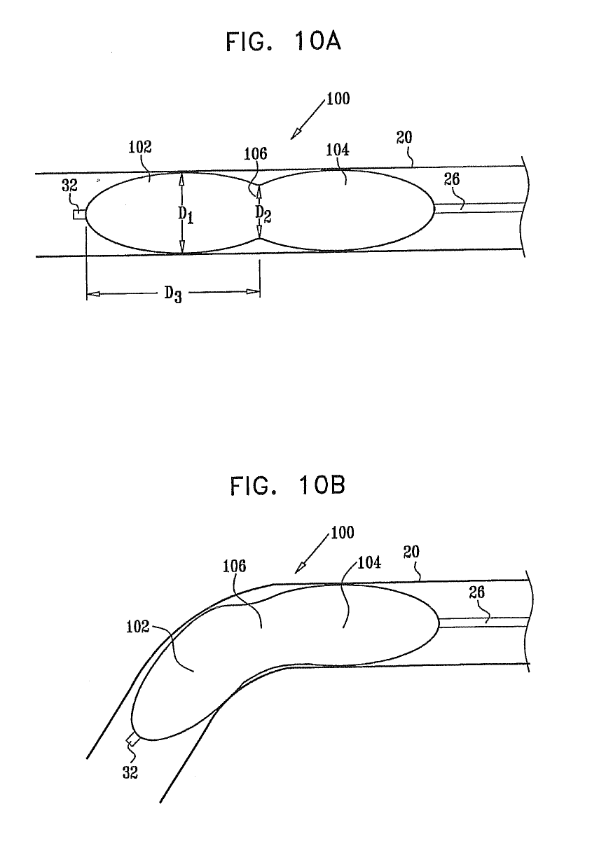

FIGS. 9A and 9B show cross-sections of the fully-inflated portion and the partially-inflated portion, respectively, in accordance with an embodiment of the present invention. Resistance of lumen 20 to radial expansion prevents the entire piston head from fully inflating (e.g., as shown in FIG. 7). Thus, partially-inflated portion 84 typically becomes somewhat wrinkled along the length of its contact with lumen 20.

Inflatable piston head 80 is regulated to respond to changes in the diameter of lumen 20 by inflating more as the lumen diameter increases, and by deflating as the lumen diameter decreases, all while maintaining satisfactory contact with the lumen. Since inflatable piston head 80 is typically made of a substantially inelastic material, a relatively modest pressure is needed to inflate the piston head. The inflation pressure is chosen to maintain an appropriate seal between the piston head and the lumen, without undue pressure on the lumen.

FIGS. 10A and 10B are pictorial illustrations of a multi-lobed piston head 100 for use in body lumen 20, constructed and operative in accordance with an embodiment of the present invention. Except for differences as noted, apparatus and techniques described hereinabove with respect to other piston heads are typically adapted for use with piston head 100.

Piston head 100 comprises a distal lobe 102 and a proximal lobe 104. Lobes 102 and 104 articulate at an intermediate portion 106. In an embodiment, dimensions of piston head 100 include: (a) a diameter D1 of distal lobe 102, which is substantially equal to the diameter of lumen 20, so as to make a satisfactory seal therewith, (b) a diameter D2 of intermediate portion 106, ranging from about 10% to 40% of D1, and (c) a length D3 of distal lobe 102, ranging from about 3 to 5 cm. It is noted that although multi-lobed piston head 100 only comprises two lobes, the scope of the present invention includes multi-lobed piston heads having more lobes (e.g., 3, 4, or 5 lobes).

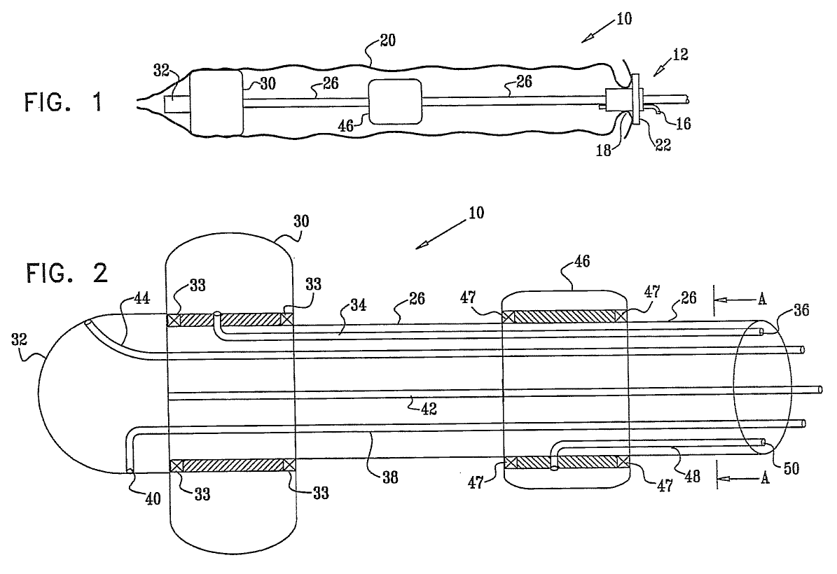

Distal and proximal lobes 102 and 104 are in fluid communication with each other through intermediate portion 106. In steady state, as well as at the levels of movement typically encountered during advancement through the colon, the pressure within lobe 102 is substantially the same as the pressure within lobe 104. Thus, passageway 34 and fluid pressure source 36 (FIG. 2) regulate the pressure within both lobes substantially simultaneously. The diameters of the two lobes, however, typically vary independently, in response to changes in the shape of lumen 20 adjacent to each of the lobes. Typically, as with all of the inflatable piston heads described herein, fluid is actively added to or removed from the piston head to maintain a generally constant pressure within the piston head.

In an embodiment of the present invention, piston head 30 and/or carrier 26 of system 10 and/or system 68 comprises a low friction coating, which acts to reduce the friction between piston head 30 and lumen 20, thereby easing the movement of piston head 30 and/or carrier 26 in lumen 20. For example, piston head 30 and/or carrier 26 may comprise a biocompatible low friction coating. Alternatively or additionally, piston head 30 and/or carrier 26 comprises a hydrophilic coating. Additionally or alternatively, the low friction coating comprises a suitable lubricant.

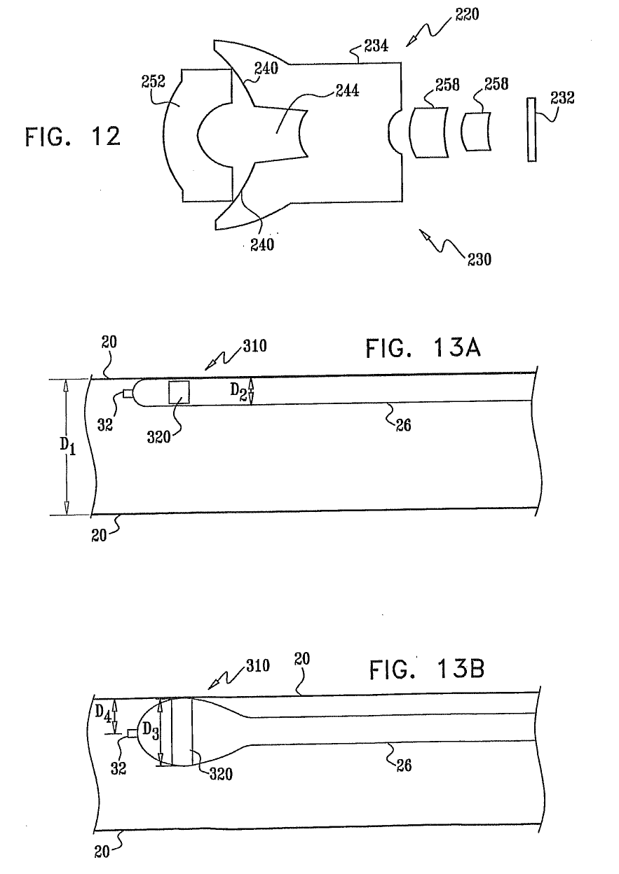

FIGS. 11A and 11B are pictorial illustrations of multi-lobed piston head 100, in accordance with an embodiment of the present invention. In FIGS. 11A and 11B, the following tubes described hereinabove are shown:

second passageway 34 in fluid communication with both lobes 102 and 104 of piston head 100, connected to source of fluid pressure 36;
vent tube 38, passing through lobes 102 and 104 of piston head 100, and having opening 40 distal to piston head 100 through which fluid is ventable to the outside;
a fluid supply tube 44, passing through piston head 100, for cleaning the area near image-capturing device 32, or in combination with vent tube 38, for cleaning body lumen 20 itself.

Second passageway 34, vent tube 38, and fluid supply tube 44 are typically flexible, which allows for the bending of piston head 100, as shown in FIG. 11B.

FIG. 12 is a schematic cross-sectional illustration of an optical system 220, in accordance with an embodiment of the present invention. For some applications, image-capturing device 32 comprises optical system 220. Optical system 220 comprises an optical assembly 230 and an image sensor 232, such as a CCD or CMOS sensor.

Optical system 220 is typically configured to enable simultaneous forward and omnidirectional lateral viewing. Light arriving from the forward end of an optical member 234, and light arriving from the lateral surface of the optical member travel through substantially separate, non-overlapping optical paths. The forward light and the lateral light are typically (but not necessarily) processed to create two separate images, rather than a unified image. For some applications, the forward view is used primarily for navigation within a body region, while the omnidirectional lateral view is used primarily for inspection of the body region.

Optical assembly 230 comprises, at a distal end thereof, a convex mirror 240 having a rotational shape that has the same rotation axis as optical member 234. Optical member 234 is typically shaped so as to define a distal indentation 244 at the distal end of the optical member, i.e., through a central portion of mirror 240. Alternatively, optical member 234 is shaped without indentation 244, but instead mirror 240 includes a non-mirrored portion in the center thereof.

Typically, optical assembly 230 further comprises a distal lens 252 that has the same rotation axis as optical member 234. For some applications, optical assembly 230 further comprises one or more proximal lenses 258, e.g., two proximal lenses 258. Proximal lenses 258 are positioned between optical member 234 and image sensor 232, so as to focus light from the optical member onto the image sensor.

For some applications, optical system 220 is configured to enable omnidirectional lateral viewing, without enabling forward viewing.

For some applications, a hydrophobic coating is applied to one or more of the transparent surfaces of optical assembly 220 that are in contact with body lumen 20.

Techniques described herein may be performed in combination with techniques described in U.S. Provisional Patent Application 60/571,438, filed May 14, 2004, entitled, “Omnidirectional and forward-looking imaging device,” which is assigned to the assignee of the present application and is incorporated herein by reference.

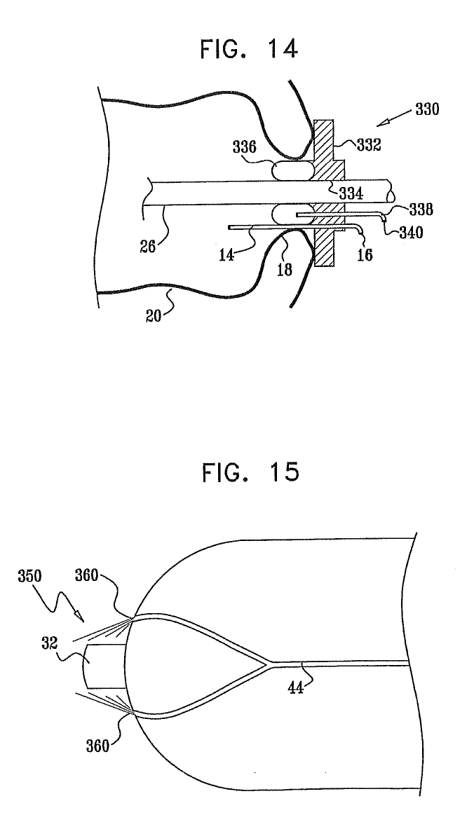

Reference is now made to FIGS. 13A and 13B, which are pictorial illustrations of a system 310 (not to scale), in accordance with an embodiment of the present invention. System 310 is generally similar to system 10 and/or system 68, except as described hereinbelow. Image-capturing device 32 of system 310 typically comprises optical system 220, described hereinabove with reference to FIG. 12, or another omnidirectional imaging device. System 310 is typically advanced distally into lumen 20 using techniques described hereinabove with reference to systems 10 and/or 68.

System 310 is withdrawn in a proximal direction by: (a) inflating lumen 20, using conventional inflation techniques for withdrawing endoscopes, and (b) pulling carrier 26 in a proximal direction. During withdrawal, the distal end of the system sometimes comes near or in contact with the wall of lumen 20, as shown in FIG. 13A. For example, lumen 20 may be inflated to a diameter D1 of between about 40 and about 70 mm, and system 310 may have an initial distal diameter D2 in a vicinity of imaging-capturing device 32 of between about 8 and about 15 mm. When system 310 is near the wall of lumen 20, the distance between the lateral portion of optical system 220 of image-capturing device 32 may be less than the minimum focal length necessary for clear omnidirectional lateral viewing.

System 310 comprises an inflation element 320, which is adapted to increase the distal diameter of system 310 from D2 (FIG. 13A) to D3 (FIG. 13B). D3 is typically between about 30 and about 45 mm. This increased distal diameter ensures that image-capturing device 32 is a distance from the wall of lumen 20 sufficient to enable focusing of the omnidirectional lateral image. For example, this increased distal diameter may ensure that a central axis of image-capturing device 32 is at least a distance D4 of 15 mm from the wall of lumen 20. For some applications, inflation element 320 comprises a sponge, which expands, for example, when exposed to liquid. Alternatively, inflation element 320 comprises a set of inflatable or expandable rings. Further alternatively, inflation element 320 comprises an inflatable balloon, which is typically contained within the body of system 310.

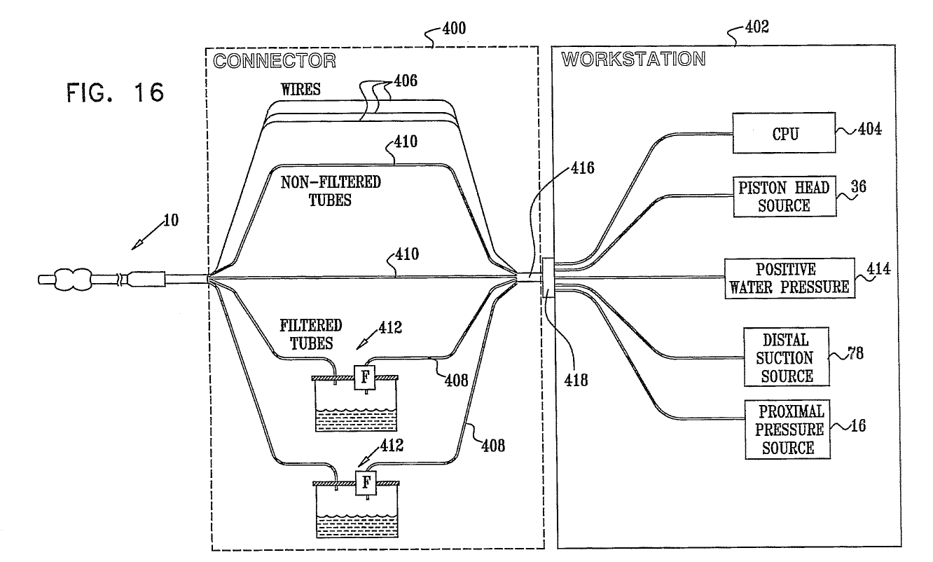

Reference is now made to FIG. 14, which is a schematic illustration of an inserter 330 for use with system 10 and/or system 68, in accordance with an embodiment of the present invention. Inserter 330 is adapted to be at least partially insertable into proximal opening 18 (e.g., the rectum) of body lumen 20 (e.g., the colon). Inserter 330 typically comprises an annular ring 332 for abutting against proximal opening 18, and an annular balloon 336 that is coupled to ring 332. Ring 332 and balloon 336 are shaped so as to define a bore 334 through which carrier 26 is arranged for sliding movement. Balloon 336 expands to form a seal between the balloon and the wall of lumen 20 in a vicinity of proximal opening 18, thereby helping maintain positive pressure created within body lumen 20.

Inserter 330 comprises first passageway 14 connected to fluid pressure source 16 (as described hereinabove with reference to FIGS. 1-3, for example), and a tube 338 for applying a positive pressure to inflate balloon 336. Tube 338 is connected to a fluid pressure source 340, which may comprise a powered fluid pressure source. (such as is available in an operating room) or a manually-operated fluid pressure source (such as a syringe). When fluid pressure source 340 comprises a syringe, the syringe is typically removed after balloon 336 has been inflated, and tube 338 and/or balloon 336 is sealed to maintain the pressure, e.g., using a check valve (valve not shown). For some applications, pressure source 16 and pressure source 340 are derived from a common fluid pressure source.

Reference is now made to FIG. 15, which is a schematic illustration of a cleaning system 350 for use with system 10 and/or system 68, in accordance with an embodiment of the present invention. Cleaning system 350 is shaped to define one or more openings 360 (e.g., between about 4 and about 10) coupled to fluid supply tube 44. Openings 360 are disposed circumferentially about the distal end of carrier 26, and oriented so that they spray at least a portion of image-capturing device 32. For applications in which image-capturing device 32 comprises optical system 220, as described hereinabove with reference to FIG. 12, openings 360 are typically oriented to spray at least a portion of the lateral omnidirectional portion of optical assembly 230, and, optionally, a portion of the distal forward portion of the assembly. For some applications, openings 360 are positioned at a circumferential angle, so as to create a vortex around image-capturing device 32.

Reference is now made to FIG. 16, which is a schematic illustration of a disposable connector 400, in accordance with an embodiment of the present invention. Disposable connector 400 is adapted to couple system 10 to an external workstation 402. In an embodiment, system 10 is also intended for single use. Workstation 402 typically comprises a control unit 404, which is typically a general-purpose CPU, and one or more sources of fluid (i.e., liquid or gas) positive and/or negative pressure, as described hereinbelow. Disposable connector 400 comprises one or more connecting elements that couple system 10 to workstation 402. The connecting elements typically include one or more electrical wires 406, one or more filtered tubes 408, and one or more non-filtered tubes 410. These connecting elements are typically, but not necessarily, coupled to a single plug 416, that is inserted in a single motion into an outlet 418 on workstation 402.

Filtered tubes 408 pass through one or more filters 412 in disposable connector 400. Filtered tubes 408 are typically used for transporting fluid that comes in contact with the content of body lumen 20. Filters 412 typically comprise a suction trap and/or a bacterial and/or viral filter, e.g., a disk filter with hydrophobic media. For some applications, workstation 402 comprises distal suction source 78, described hereinabove with reference to FIG. 6, and one of filtered tubes 408 is coupled to the distal suction source. Alternatively or additionally, workstation 402 comprises proximal pressure source 16, described hereinabove with reference to FIG. 3, and one of filtered tubes 408 is coupled to the proximal pressure source. Further alternatively or additionally, workstation 402 comprises one or more additional sources or positive and/or negative fluid pressure, and one of filtered tubes 408 is coupled to the additional source of fluid pressure.

Non-filtered tubes 410 are typically used for transporting fluid that does not come in contact with the content of body lumen 20, or fluid that is always tinder positive pressure. For some applications, workstation 402 comprises fluid pressure source 36, which is in fluid communication with the interior of piston head 30, as described hereinabove with reference to FIG. 6, and one of non-filtered tubes 410 is coupled to the fluid pressure source. Alternatively or additionally, workstation 402 comprises a source of positive pressure 414, for example, for irrigating body lumen 20, or cleaning tool 79, and one of non-filtered tubes 410 is coupled to the positive pressure source.

Workstation 402 typically, but not necessarily, operates independently of hospital-supplied suction and/or positive gas pressure sources. Instead, in an embodiment, an electric motor within workstation 402 generates the suction and/or positive gas pressure.

For some applications, disposable connector 400 is adapted to couple a conventional endoscope to a conventional workstation.

Although the piston head has been described in embodiments of the present invention as being in direct contact with the wall of the GI tract, the scope of the invention includes establishing contact between the piston head and the wall of the GI tract through an intermediary, such as a sheath surrounding the piston head.

Techniques described herein may be performed in conjunction with techniques described in the following patent applications, which are assigned to the assignee of the present application and are incorporated herein by reference: (a) U.S. patent application Ser. No. 10/838,648 to Gross et al., entitled, “Pressure-propelled system for body lumen,” filed May 3, 2004, and (b) PCT Patent Publication WO 2005/065044 to Cabiri et al., entitled, “Pressure-propelled system for body lumen,” filed Jan. 3, 2005.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
an elongate carrier, adapted to be inserted through a proximal opening of a body lumen;
a workstation, which comprises a generator of negative fluid pressure; and
a disposable connector, adapted to be coupled to the elongate carrier, the connector comprising a filter and a tube coupled to the filter, the tube adapted to be coupled to the negative fluid pressure.

2. The apparatus according to claim 1, wherein the filter comprises a suction trap comprising a container for containing fluid from the body lumen.

3. The apparatus according to claim 1, wherein the workstation comprises a source of positive fluid pressure, and wherein the elongate carrier comprises a piston head coupled to a distal portion of the carrier and adapted to:
form a pressure seal with a wall of the lumen after the carrier has been inserted into the lumen, and
be advanced distally through the body lumen in response to pressure from the positive fluid pressure source.

4. The apparatus according to claim 1, wherein the disposable connector is adapted to be fixedly coupled to the elongate carrier.

5. The apparatus according to claim 1, wherein the disposable connector is adapted to be removably coupled to the elongate carrier.

6. The apparatus according to claim 1, wherein the lumen includes a gastrointestinal (GI) tract, and wherein the carrier is adapted to be inserted through the proximal opening of the GI tract.

7. The apparatus according to claim 6, wherein the GI tract includes a colon, and wherein the carrier is adapted to be inserted through the proximal opening of the colon.

8. The apparatus according to claim 2, wherein the lumen includes a gastrointestinal (GI) tract, and wherein the carrier is adapted to be inserted through the proximal opening of the GI tract.

9. The apparatus according to claim 8, wherein the GI tract includes a colon, and wherein the carrier is adapted to be inserted through the proximal opening of the colon.

10. The apparatus according to claim 3, wherein the lumen includes a gastrointestinal (GI) tract, and wherein the carrier is adapted to be inserted through the proximal opening of the GI tract.

11. The apparatus according to claim 10, wherein the GI tract includes a colon, and wherein the carrier is adapted to be inserted through the proximal opening of the colon.

12. The apparatus according to claim 4, wherein the lumen includes a gastrointestinal (GI) tract, and wherein the carrier is adapted to be inserted through the proximal opening of the GI tract.

13. The apparatus according to claim 12, wherein the GI tract includes a colon, and wherein the carrier is adapted to be inserted through the proximal opening of the colon.

14. The apparatus according to claim 5, wherein the lumen includes a gastrointestinal (GI) tract, and wherein the carrier is adapted to be inserted through the proximal opening of the GI tract.

15. The apparatus according to claim 14, wherein the GI tract includes a colon, and wherein the carrier is adapted to be inserted through the proximal opening of the colon.
